(19) United States
(12) Patent Application Publication
(10) Pub. No.: US 2020/0205494 A1
(43) Pub. Date: Jul. 2, 2020

(54) COOLING PAD FOR NECK WITH EYEGLASS CORD FOR GLASSES

(71) Applicant: Irene Marlene Lucero, Albuquerque, NM (US)

(72) Inventor: Irene Marlene Lucero, Albuquerque, NM (US)

(21) Appl. No.: 16/727,373

(22) Filed: Dec. 26, 2019

Related U.S. Application Data
(60) Provisional application No. 62/785,568, filed on Dec. 27, 2018.

(51) Int. Cl. A41D 23/00; A61F 7/00

(52) U.S. Cl. CPC A41D 23/00; A41D 2023/002; A61F 2007/0011; A61F 7/00

(57) ABSTRACT

A neck collar includes a narrow elongated base proportioned and contoured to extend circumferentially around the neck of the user. The elongated base has an inner surface in confronting engagement with the neck. The elongated base may be separated at opposing ends. The ends of base may be disposed below a user's chin when the elongated base is worn by the user. The neck collar includes a cooling composition disposed within an outer fabric covering. The neck collar also includes an additional loop on both ends to attach an eyeglass cord.

### CROSS-REFERENCE TO RELATED APPLICATION

This application claims the benefit of priority of U.S. provisional application No. 62/785,568, filed Dec. 27, 2018, the contents of which are herein incorporated by reference.

### BACKGROUND OF THE INVENTION

The present invention relates to cooling pads and, more particularly, to a cooling pad that is shaped to fit and retain around a user's neck.

As is known, cooling or refreshing bags and/or pads have been widely used to cool down individuals during heat stress and/or to treat disorders, injury, and fatigue. Such bags and/or pads generally contain chemical components. Cold compresses of cloth or the like soaked with water or an evaporating chemical are also known. When used for these purposes, such cooling bags and pads are directly applied to the body. Current cooling bags do not easily secure to a user's neck.

As can be seen, there is a need for a cooling pad that is shaped to fit and retain around a user's neck.

### SUMMARY OF THE INVENTION

In one aspect of the present invention, a therapeutic wearable device comprises: a neck collar comprising an elongated base contoured to extend circumferentially around a neck of a user, the elongated base comprising a first end opposite a second end, wherein a first fastening loop is coupled to the first end and a second fastening loop is coupled to the second end, the neck collar further comprising a cooling pad including a cooling composition capable of generating a cold temperature for a period of time; and at least one cord comprising a first end having a first hook and a second end having a second hook, wherein the first hook releasably attaches to the first fastening loop and the second hook releasably attaches to the second fastening loop.

In another aspect of the present invention, a method of using a therapeutic device comprises steps of: wrapping a neck collar around a neck, the neck collar comprising an elongated base comprising a first end opposite a second end, wherein a first glasses connector loop is secured to the first end and a second glasses connector loop is secured to the second end, the neck collar further comprising a cooling pad including a cooling composition capable of generating a cold temperature for a period of time; attaching a first hook of a first glasses cord to the first glasses connector loop and attaching a second hook of the first glasses cord to a pair of glasses; and attaching a first hook of a second glasses cord to the second glasses connector loop and attaching a second hook of the second glasses cord to the pair of glasses.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention;

FIG. 2 is a front view of an embodiment of the present invention;

FIG. 3 is a side view of an embodiment of the present invention; and

FIG. 4 is a front view of an embodiment of the present invention, illustrated in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

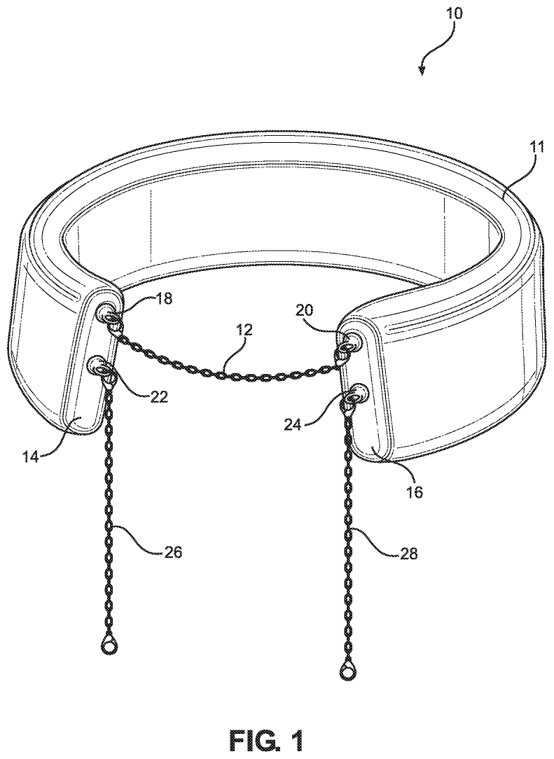

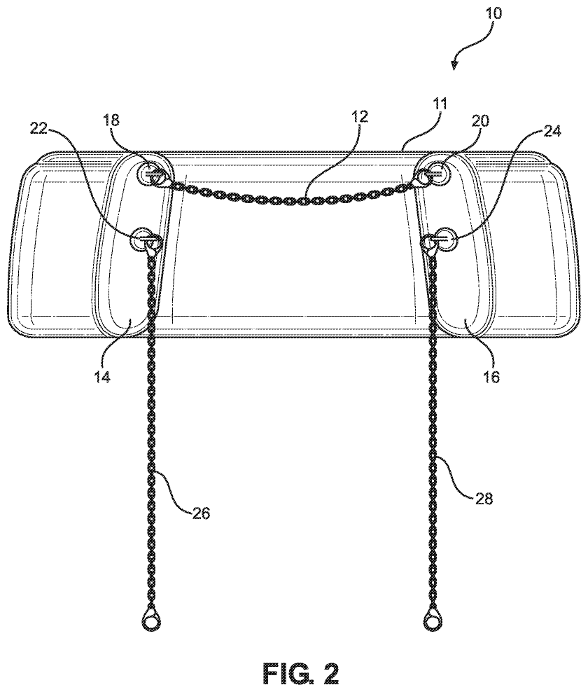

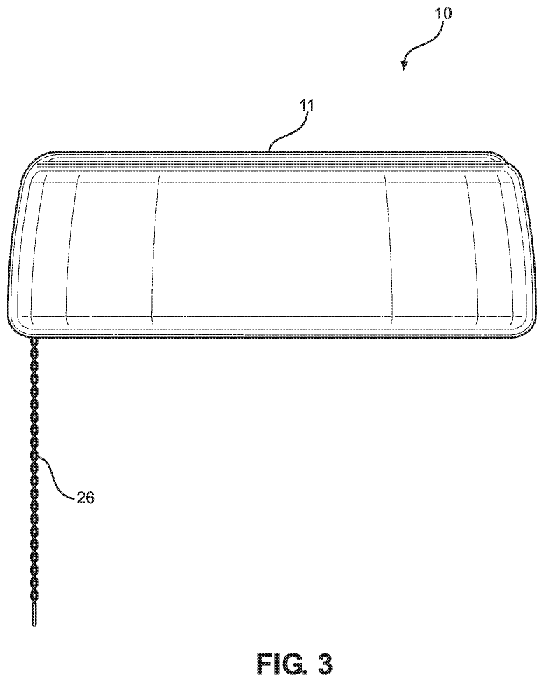

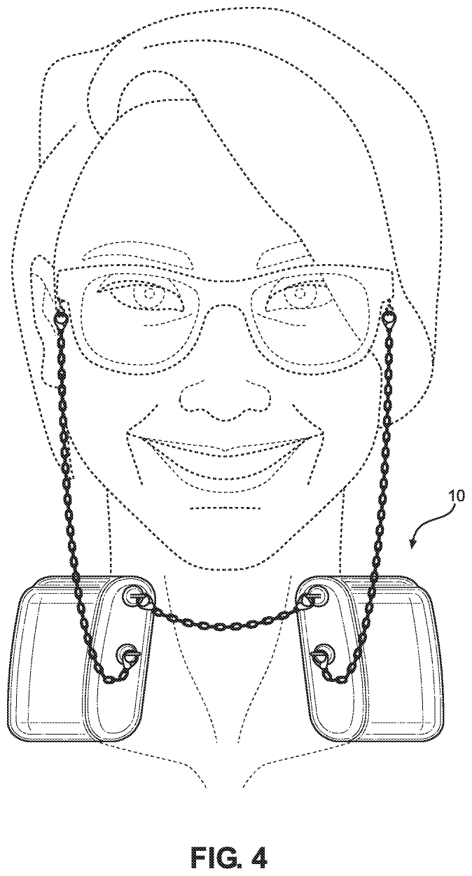

Referring to FIGS. 1 through 4, the present invention includes a neck collar 10. The neck collar 10 includes a narrow elongate base 11 proportioned and contoured to extend circumferentially around the neck of the user. The elongated base 11 has an inner surface in confronting engagement with the neck. The elongated base 11 may be separated at opposing ends. The ends of base 11 may be disposed below a user's chin when the elongated base 11 is worn by the user.

The neck collar 10 may include a cooling pad having a cooling composition. The cooling pad is disposed within an outer fabric covering. A wide variety of suitable cooling compositions may be used herein. The cooling composition may include a plurality of cooling polymer beads, ice cubes, cold-water, heat-storing antifreeze solution, polymeric cold-retaining agents, and endothermic agents, and are packed in a water-impermeable bag or other such receptacle. Water-based compositions such as ice cubes and cold-water are, of course, re-useable. For example, such water-based compositions can be refrigerated after the previous use.

A connector is provided for securely fastening the neck collar 10 about the neck. A first fastening loop 18 may be secured to a first end 14 of the neck collar 10 and a second fastening loop 20 may be secured to a second end 16 of the neck collar 10. A fastening cord 12 may include a first end with a first hook and a second end with a second hook. The first hook may releasably couple to the first fastening loop 18 and the second hook may releasably couple to the second fastening loop 20, connecting the first end 14 to the second end 16 and thereby connecting the neck collar 10 to the user's neck.

The present invention may further include a glasses connector 26, 28. In such embodiments, a first glasses connector loop 22 may be secured to the first end 14 of the neck collar 10 and a second glasses connector loop 24 may be secured to the second end 16 of the neck collar 10. A first glasses cord 26 may include a first end with a first hook and a second end with a second hook. The first hook may releasably couple to the first glasses connector loop 22 and the second hook may releasably couple to glasses. A second glasses cord 28 may include a first end with a first hook and a second end with a second hook. The first hook may releasably couple to the second glasses connector loop 28 and the second hook may releasably couple to glasses. The first glasses cord 26 and the second glasses cord 28 releasably secure glasses to the neck collar 10, thereby connecting the glasses to the user.

The fastening cord 12 may be a chain, fabric, or elastic cord. Likewise, the first glasses cord 26 and the second glasses cord 28 may be a chain, fabric, or elastic cord. The first hook and the second hook of the fastening cord 12, the first glasses cord 26, and the second glasses cord 28 may include a regular hook or may include a spring hook, such as a toggle clasp or lobster clasp. Opposing ends of the first glasses cord 26 and the second glasses cord 28 may include a rubber loop, which can then be placed on the arms of user's glasses. The glasses may then drape down from the collar in a comfortable fashion.

In the use, the neck collar 10 is cooled in a refrigerator or by cold water. The neck collar 10 is then placed about the neck with the base 11 inwardly facing. The neck collar 10 thereby cools the user's neck. The fastening cord 12 may attach the ends 14, 16 together, thereby securing the neck collar 10 to the user. The user may then attach either reading glasses or sunglasses to the neck collar 10 via the first glasses cord 26 and the second glasses cord 28. The cooling properties of the neck collar 10 protect a user while exercising, against heat, hot flashes, and sun stroke as well as providing a comfortable way of holding eye wear.

It should be understood, of course, that the foregoing relates to exemplary embodiments of the invention and that modifications may be made without departing from the spirit and scope of the invention as set forth in the following claims.

What is claimed is:

1. A therapeutic wearable device comprising:
a neck collar comprising an elongated base contoured to extend circumferentially around a neck of a user, the elongated base comprising a first end opposite a second end, wherein a first fastening loop is coupled to the first end and a second fastening loop is coupled to the second end, the neck collar further comprising a cooling pad including a cooling composition capable of generating a cold temperature for a period of time; and
a fastening cord comprising a first end having a first hook and a second end having a second hook, wherein the first hook releasably attaches to the first fastening loop and the second hook releasably attaches to the second fastening loop.

2. The therapeutic wearable device of claim 1, wherein the neck collar comprises an outer fabric layer and the cooling pad is disposed inside the outer fabric layer.

3. The therapeutic wearable device of claim 1, wherein the cooling pad comprises a cooling composition comprising at least one of a plurality of polymer beads, ice cubes, cold-water, heat-storing antifreeze solution, a polymeric cold-retaining agents, and endothermic ingredients.

4. The cooling pad of claim 1, wherein a first glasses connector loop is secured to the first end and a second glasses connector loop is secured to the second end.

5. The cooling pad of claim 4, further comprising a first glasses cord having a first end with a first hook and a second end with a second hook, and a second glasses cord having a first end with a first hook and a second end with a second hook, wherein the first hook of the first glasses cord releasably attaches to the first glasses connector loop and the first hook of the second glasses cord releasably attaches to the second glasses connector loop.

6. A method of using a therapeutic device comprising steps of:
wrapping a neck collar around a neck, the neck collar comprising an elongated base comprising a first end opposite a second end, wherein a first glasses connector loop is secured to the first end and a second glasses connector loop is secured to the second end, the neck collar further comprising a cooling pad including a cooling composition capable of generating a cold temperature for a period of time;
attaching a first hook of a first glasses cord to the first glasses connector loop and attaching a second hook of the first glasses cord to a pair of glasses; and
attaching a first hook of a second glasses cord to the second glasses connector loop and attaching a second hook of the second glasses cord to the pair of glasses.

7. The method of claim 6, wherein the neck collar comprises an outer fabric layer and the cooling pad is disposed inside the outer fabric layer.

8. The method of claim 6, wherein the cooling pad comprises a cooling composition comprising at least one of a plurality of polymer beads, ice cubes, cold-water, heat-storing antifreeze solution, a polymeric cold-retaining agents, and endothermic ingredients.

9. The method of claim 6, wherein a first fastening loop is coupled to the first end of the neck collar and a second fastening loop is coupled to the second end of the neck collar.

10. The method of claim 9, further comprising steps of:
attaching a first hook of a fastening cord to the first fastening loop; and
attaching a second hook of the fastening cord to the second fastening loop.
